(19) United States
(12) Patent Application Publication
(10) Pub. No.: US 2023/0181381 A1
(43) Pub. Date: Jun. 15, 2023

(54) A SWAB

(71) Applicant: NATIONAL UNIVERSITY OF SINGAPORE, Singapore (SG)

(72) Inventor: Yin Chiang BOEY, Singapore (SG)

(21) Appl. No.: 17/924,855

(22) PCT Filed: May 19, 2021
(86) PCT No.: PCT/SG2021/050271

§ 371 Date: Nov. 11, 2022

(30) Foreign Application Priority Data
May 19, 2020 (SG) 10202004676W

(51) Int. Cl. A61F 13/38; A61B 10/00

(52) U.S. Cl. CPC A61F 13/38; A61B 10/0045; A61B 10/0233

(57) ABSTRACT

Described is a swab comprising a rod having a longitudinal axis and a tip. The tip includes a proximal region at a distal end of the rod, a distal region opposite the proximal region, and a plurality of straight blades extending from the proximal region to the distal region, parallel to the longitudinal axis.

### TECHNICAL FIELD

The present invention relates to a swab for collecting a sample from a body cavity, such as a nasopharyngeal swab.

### BACKGROUND

Devices, such as swabs, for collecting biological specimens of organic material are known in the field of clinical and diagnostic analyses. These swabs generally include a cylindrical rod or stick containing on a collection end or tip formed from a wad of fiber material, such as rayon or a natural fiber such as cotton. The tip has hydrophilic properties to allow rapid absorption of the specimen from a body cavity. Stable adherence of the fiber wrapped around the end or tip of the rod or stick is generally achieved by gluing.

Collection swabs containing the collected material are often immersed in a viral transport medium in a receptacle such as a test tube, vial, culture dish, or culture bottle, soon or immediately after collection. This is to preserve and conserve the collected specimen during storage and/or transport to, for example, a laboratory.

Given the essentially random arrangement of fibres in the tip, the fibres may block part of the sample from release into the viral transport medium. The tip may also entrain air which can further inhibit release of the sample. Since the amount of the sample released into the viral transport medium is unpredictable.

It would be desirable to overcome or ameliorate at least one of the above-described problems, or at least to provide a useful alternative.

### SUMMARY

Described herein is a swab comprising:
a rod having a longitudinal axis; and
a tip comprising:
   a proximal region at a distal end of the rod;
   a distal region opposite the proximal region; and
   a plurality of straight blades extending from the proximal region to the distal region.

The blades may extend in a direction parallel to the longitudinal axis. The blades may be spaced to define a plurality of reservoirs therebetween. Each reservoir may be defined by a side surface of each of two blades, the blades being sufficiently nearby to generate surface tension to retain the sample in the reservoir. The reservoirs may be non-contiguous.

The tip may comprise a support extending from the proximal region to the distal region, and the plurality of blades extend outwardly of the support. The support may comprise a central shaft. The blades may extend radially outwardly from the central shaft.

The support may comprise a flat leaf. The blades may project in a direction normal to a plane of the support. The support may form two of said blades, each blade comprising one of two opposite, longitudinally extending edges of the flat leaf. The tip may comprise, on each side of the support, either: three blades extending normal to a plane of the support; or four blades extending normal to a plane of the support.

At least one of the blades may comprise a relief for increasing flexibility of the tip.

The blades may taper towards the distal region.

The proximal region may comprise a fluted section extending from a first diameter at the rod to larger, second diameter at the blades.

All edges of the tip may be rounded.

The blades may be rounded to form a hemispherical tip.

The rod may comprise a handling section and a tapered section extending distally of the handling section.

The rod may comprise a weakened section in the rod, spaced from the tip. The weakened section may be in the handling section.

The tip may have a diameter of about 3.0 mm or 3.5 mm.

Advantageously, the blades extend from the proximal region to the distal region, parallel to the longitudinal axis. Once the tip has been inserted into the orifice, it is rotated about the longitudinal axis. The outer edges of the blades therefore extend parallel to the axis of rotation and perpendicular to the direction of motion of those edges against the internal wall of the body cavity. This can increase the amount of sample scraped from the internal wall of the body cavity, for each rotation.

Advantageously, by rounding the edges of the tip there is increased comfort to the subject from whom the sample is being taken. The tip will not scratch the internal wall of the orifice from which the sample is being taken.

Advantageously, by providing a weakened section in the rod, the rod can be snapped at the weakened section, to fit into a sample collection tube or receptacle. In some embodiments, the weakened section may be spaced from the tip by an amount designed to conform to the end of the tube or receptacle. If the length of the tip and portion of the rod distal of the weakened section is roughly equal to, but slightly less than, the length of the internal volume of the sample collection tube or receptacle, the tip will remain in the viral transport medium even if the tube or receptacle bounces around or is angled, e.g., while in transit.

### BRIEF DESCRIPTION OF THE DRAWINGS

Embodiments of the present invention will now be described, by way of non-limiting example, with reference to the drawings in which:

FIG. 1 is a side view of swab in accordance with present teachings;

FIGS. 2 to 4 are a cross-section view, top view and end view respectively of the swab of FIG. 1;

FIG. 5 is a perspective view of the tip of the swab of FIG. 1;

FIG. 6 is an end view of the tip of an alternative embodiment of a swab in accordance with present teachings;

FIG. 7 is an end view of the tip of an alternative embodiment of a swab in accordance with present teachings;

FIG. 8 is a perspective view of the tip of FIG. 7;

FIG. 9 is a perspective view of another embodiment of a swab in accordance with present teachings, showing radially extending blades; and

FIG. 10 is a perspective view of the tip of the swab of FIG. 9; and

FIG. 11 is a partial close-up view of the tip of the swab of FIG. 9.

### DETAILED DESCRIPTION

Described herein are swabs for use in the collection of a sample from the body cavity of subject. The swabs have a highly regularised, open structure. That structure is designed to retain the sample—e.g. under surface tension in reservoirs in the tip—while facilitating ready release upon immersion of the tip in a viral transport medium (VTM).

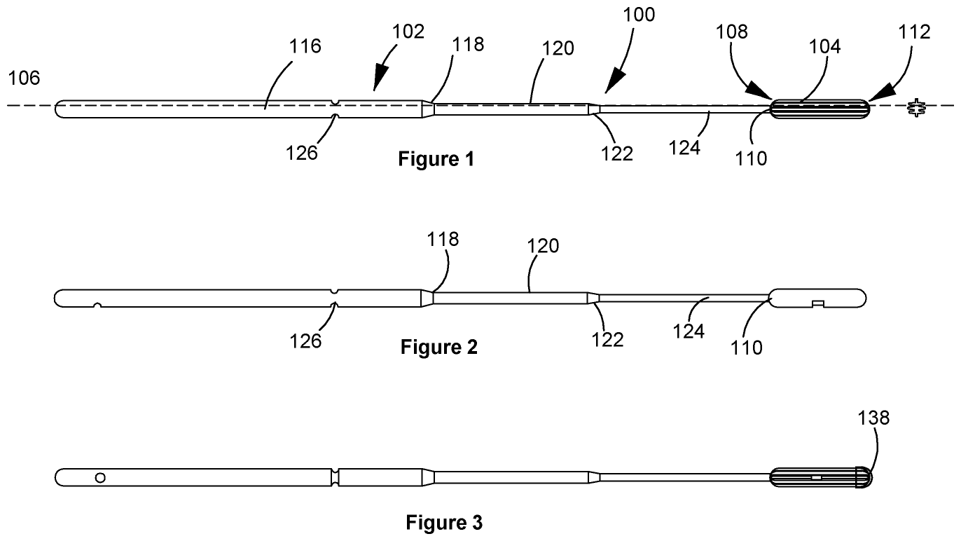

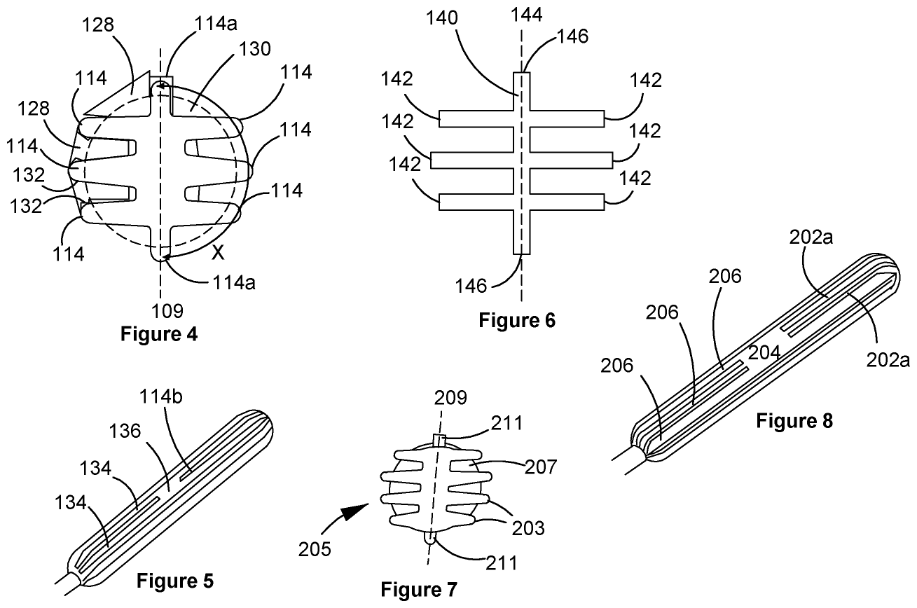

Such a swab 100, is shown in FIG. 1. The swab 100 includes a rod 102 and a tip 104. The rod 102 has a longitudinal axis 106. The tip 104 has a proximal region 108 at a distal end 110 of the rod 102, and a distal region 112 opposite the proximal region 108. The tip 104 further includes a plurality of straight blades 114 (which also includes blades 114a and 114b—see FIGS. 2 and 4) extending from the proximal region 108 to the distal region 112.

In general, the rod 102 will be formed in a cylindrical shape. The longitudinal axis 106 passes through the centre of the cylinder. The rod 102 comprises a handling section 116 and a tapered section 118. The tapered section 118 extends distally of the handling section 116. As shown with reference to cross-section FIG. 2, the tapered section 118 extends from the larger diameter handling section 116 to an intermediate diameter section 120. In the present embodiment, the rod 102 includes a further tapered section 122 extending from the intermediate diameter section 120 to a narrow diameter section 124 that includes the distal end 110 of the rod 102.

The reduction in the diameter between the handling section 116 and the distal end 110 increases the flexibility. The flexibility reduces the force applied to the internal surface of the body cavity (e.g. nostril, mouth, rectum or other orifice) from where the sample was taken.

In other embodiments, the rod has a consistent diameter along its length, or any other number of reductions in diameter.

The rod 102 also includes a weakened section 126, spaced from the tip 104. After the tip 104 has been placed into VTM—e.g. in a tube or other receptacle—the weakened section 126 enables the rod 102 to be readily snapped so that the tip 104 remains in the VTM. To that end the weakened section 126 is spaced from the distal region 112 by a distance just less than the internal length of the receptacle—presently, the weakened section 126 is in the handling section 116 but it may instead be located between the handling section 116 and the distal end 110. Accordingly, if the receptacle is jolted or tilted while being moved, the tip 104 will remain in the VTM.

As mentioned above, the tip 104 includes a plurality of straight blades 114. The blades 114 may extend parallel to the longitudinal axis 106 from the proximal region 108 to the distal region 112. Alternatively, the blades 114 may extend at an angle to the longitudinal axes 106, from the proximal region 108 to the distal region 112.

The blades 114 are spaced (i.e. from each other) to define a plurality of reservoirs 128 therebetween. The reservoirs 128 are defined by the side surfaces of the blades. As used herein, the term “defined by the side surfaces of the blades” includes the reservoirs 128 being solely defined by the blades 114—e.g. the sides of neighbouring blades may meet to define, for example, a reservoir with a triangle cross-section—and the reservoirs 128 being defined by the blades 114 and other components such as support 130, some of the reservoirs 128 being identified in FIG. 4. A broken line circle has been placed over support 130 to show more clearly where the support 130 provides the base of each reservoir 128.

In the embodiment shown in FIG. 4, each reservoir is defined by a side surface 132 of each of two blades 114. The reservoirs 128 hold the sample taken from the body cavity. Due to the proximity of the side surfaces 132 of the blades 114, the sample is retained in the reservoir 128 by surface tension. The blades 114 are therefore sufficiently nearby (i.e. near each other) to generate surface tension to retain the sample in the reservoir.

Each blade 114 forms a solid rib extending along the tip 104. This separates the reservoir 128 on one side of a blade 114 from the reservoir 128 on the other side of the blade 114, such that some reservoirs 128 are non-contiguous. In other cases, reservoirs such as reservoirs 134 of FIG. 5 may be connected. The blade 114b, includes a relief 136 for increasing flexibility of the tip 104 though, in some embodiments, no such relief may be necessary. In other embodiments, conduits may be provided through the blades, between the reservoirs such that their volumes are connected.

The support 130 is for supporting the blades 114. The support 130 extends from the proximal region 108 to the distal region 112, and each blade 114 extends outwardly of the support 130. In the embodiment shown in FIG. 4, the blades 114 are parallel, with the exception of top and bottom blades 114a that are themselves parallel but are otherwise perpendicular to the other blades.

The support 130 comprises or forms a central shaft. As such, it is cylindrical. The blades 114 therefore form shallow ribs or ridges protruding from the surface of the central shaft. In other embodiments, such as that shown in FIG. 6, the support 140 may be a flat leaf or planar shape. The blades 142 may extend in a direction normal to a plane 144 of the flat leaf 140 in a similar manner to the blades 114 (i.e. in parallel). The flat leaf 140 may also form two blades 146, each blade 146 being one of two opposite, longitudinally extending edges of the flat leaf 140.

To maintain comfort for the subject during sample collection, all edges of the tip 104 are rounded. This reduces scratching by the blades on the internal wall of a body cavity. In addition, as shown in FIGS. 1 to 3, the blades 114 form a hemispherical tip 138 at the distal region 112.

As shown in FIGS. 1 to 5, the blades 114 (including 114a and 114b) extend parallel to the longitudinal axis 106 of the swab 100. During sample collection, the swab 100 will typically be rotated about that axis 106. This results in the blades 114 being drawn in the direction of arrow X (see FIG. 4) along the internal wall of the body cavity. The blades 114 therefore scrape the sample from the internal wall of the body cavity. The blades extend perpendicular to the direction of motion during rotation about axis 106. As such the full length of each blade 114 that is in contact with the internal wall of the body cavity may contact that wall to scrape the sample therefrom. This results in very fast sample collection and reduced discomfort to the subject when compared with cotton or flocked swabs.

The swab 200, shown in FIGS. 7 and 8 is the same as that shown in FIGS. 1 to 5, except that there are more blades 202 on swab 200 than on swab 100. In addition, the relief 204 extends through two blades 202a, rather than a single blade 114b, and thus merges three reservoirs 206 rather than two reservoirs 134.

In each case, the relief 136, 204 may also be used to facilitate injection moulding the swab 100, 200. The relief 136, 204 may form an ejection site for the mould used for injection moulding. A further ejection site may be provided towards a proximal end of the rod (i.e. the end opposite the tip).

The swabs 100, 200 may come in various sizes to suit different sized orifices of body cavities from which the samples are to be collected. There may be any number of blades on a swab of a particular size, depending on the desired reservoir size, the comfort of the subject and other considerations. For example, the tip 104 may comprise three blades 114 projecting from either side of the support 130, or four blades 203 projecting from either side of the support 207 (per tip 205), normal to a plane of the support 130, 209. Note, the plane 109, 209 of the present embodiments extends through two further blades 114a, 211 of the support 130,

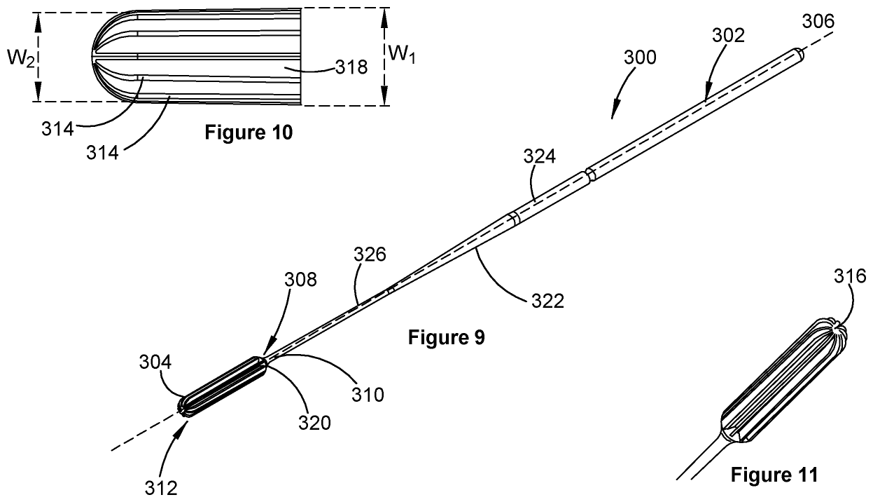

The swab 300 of FIGS. 9 to 11 is also similar in many respects to swabs 100, 200. The swab 300 includes a rod 302 and a tip 304. The rod 302 has a longitudinal axis 306. The tip 304 has a proximal region 308 at a distal end 310 of the rod 302, and a distal region 312 opposite the proximal region 308. The tip 304 further includes a plurality of straight blades 314 extending from the proximal region 308 to the distal region 312, parallel to the longitudinal axis 306.

While each of the blades 314 extends from the proximal region 308 of the tip 304 to the distal region 312, the blades 314 extend radially outwardly from the axis 306 of the swab 300. In this context, “from the axis” refers to the direction of extent of the blades 314 rather than the blades 314 terminating at or intersecting the axis 306. Instead, the blades 314 are all mounted to a central shaft 316.

By extending the blades radially, the size of the reservoirs 318 formed between the blades is uniform. In addition, the reservoirs 318 of swab 300 are deeper than those of swab 100 since the central shaft 316 is of smaller diameter than central shaft 130, yet the diameter of the tip 304 may be the same, smaller or even larger than that of tip 104—e.g. tip 104 may have a 3 mm diameter whereas tip 304 may have a 3.5 mm diameter. As a result, for the same tip diameter, the reservoirs 318 of swab 300 may retain a greater volume of the sample than the reservoirs 128 of swab 100.

The blades 314 are mounted, at the proximal region of the tip 304, to a fluted section 320 of the tip 304. The fluted section 320 extends from a first (smaller) diameter at the rod 302 to a (larger) second diameter at the blades 314. In addition, where rod 102 includes two, short tapers 118, 122 that reduce the diameter of the rod 102 from the larger diameter handling section 116 to the smaller diameter distal section 124, rod 304 includes a single, more gradual taper 322, extending between the larger diameter handling section 324 and the narrower diameter distal section 326.

As shown in FIG. 11, the blades 314 taper towards the distal region of the tip 304, from a first width or diameter W₁ to a smaller width or diameter w₂. The same taper may be incorporated into other embodiments taught herein. This taper makes it easier for the tip to be withdrawn from a moulding tool.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A swab comprising:
a rod having a longitudinal axis; and
a tip comprising:
a proximal region at a distal end of the rod;
a distal region opposite the proximal region; and
a plurality of straight blades extending from the proximal region to the distal region.

2. The swab of claim 1, wherein the blades are spaced to define a plurality of reservoirs therebetween.

3. The swab of claim 2, wherein each reservoir is defined by a side surface of each of two blades, the blades being sufficiently nearby to generate surface tension to retain the sample in the reservoir.

4. The swab of claim 2 or 3, wherein the reservoirs are non-contiguous.

5. The swab of any one of claims 1 to 4, wherein the tip comprises a support extending from the proximal region to the distal region, and the plurality of blades extend outwardly of the support.

6. The swab of claim 5, wherein the support comprises a central shaft.

7. The swab of claim 6, wherein the blades extend radially outwardly from the central shaft.

8. The swab of claim 5, wherein the support comprises a flat leaf.

9. The swab of claim 6 or 8, wherein the blades project in a direction normal to a plane of support.

10. The swab of claim 8 or 9, wherein the support forms two of said blades, each blade comprising one of two opposite, longitudinally extending edges of the flat leaf.

11. The swab of any one of claims 1 to 10, wherein at least one said blade comprises a relief for increasing flexibility of the tip.

12. The swab of any one of claims 1 to 11, wherein the blades taper towards the distal region.

13. The swab of any one of claims 1 to 12, wherein the proximal region comprises a fluted section extending from a first diameter at the rod to larger, second diameter at the blades.

14. The swab of any one of claims 1 to 13, wherein all edges of the tip are rounded.

15. The swab of claim 14, wherein the blades are rounded to form a hemispherical tip.

16. The swab of any one of claims 1 to 15, wherein the rod comprises a handling section and a tapered section extending distally of the handling section.

17. The swab of any one of claims 1 to 16, wherein the rod comprises a weakened section in the rod, spaced from the tip.

18. The swab of claim 16, wherein the rod comprises a weakened section in the handling section.

19. The swab of any one of claims 1 to 18, wherein the tip has a diameter of about 3.0 mm or 3.5 mm.

20. The swab of claim 9, wherein the tip comprises either:
on each side of the support, three blades extending normal to a plane of the support; or
on each side of the support, four blades extending normal to a plane of the support.

21. The swab of any one of claims 1 to 20, wherein the blades extend in a direction parallel to the longitudinal axis.
